(19) United States
(12) Patent Application Publication
(10) Pub. No.: US 2018/0216131 A1
(43) Pub. Date: Aug. 2, 2018

(54) ARTIFICIALLY SYNTHESIZED INSECT-RESISTANT PROTEIN, BIOLOGICAL MATERIALS ASSOCIATED THEREWITH, AND USE THEREOF

(71) Applicant: Institute of Cotton Research, Chinese Academy of Agricultural Sciences, Anyang, Henan (CN)

(72) Inventors: Fuguang LI, Anyang, Henan (CN); Chaojun ZHANG, Anyang, Henan (CN); Xueyan ZHANG, Anyang, Henan (CN); Ye WANG, Anyang, Henan (CN); Qianhua WANG, Anyang, Henan (CN)

(21) Appl. No.: 15/128,939

(22) PCT Filed: Mar. 27, 2015
(86) PCT No.: PCT/CN2015/000212

§ 371 Date: Sep. 23, 2016

(51) Int. Cl. C12N 15/82; C07K 14/325; A01N 63/00

(52) U.S. Cl. CPC C12N 15/8286; A01N 63/00; C07K 14/325

(57) ABSTRACT

An artificially synthesized insect-resistant protein, biological materials associated therewith, and use thereof are disclosed. The protein is designated as MRP001 protein, and is a protein of A1) or A2): A1) a protein having an amino acid sequence as shown in SEQ ID No.6; and A2) a protein having insect resistance derived from A1) by substituting, and/or deleting, and/or adding one or more amino acid residues in the amino acid sequence of the protein of A1). It is confirmed through experiments that the MRP001 protein and biological materials associated therewith have resistance to plant bug, and are useful in the preparation of biological ant-insect agent containing the protein, and in the cultivation of insect-resistant transgenic cotton, fruits, tea, rice, vegetables and other crops, thereby reducing the application of pesticides and alleviating the environmental pollution, and thus being of great economic value and broad application prospect.

### BACKGROUND

### Technical Field

The present invention relates to an artificially synthesized insect-resistant protein, biological materials associated therewith, and use thereof, which fall within the technical field of genetic engineering and control of agricultural pests.

### Related Art

China is the largest producer and consumer of cotton, and also the largest producer and exporter of textiles and apparel in the world. The total production of cotton accounts for about 25% of the global production. The rise and decline of cotton production has an impact on the development of China's national economy and the improvement of people's living standards. In China, the cotton planted area is 6,000,000-8,300,000 hectares, which is ⅕-⅙ of the total cotton planted area and ranks the second leading position in the world. The annual total production of lint cotton is about 6,000,000 tons, which accounts for about ¼ of the global production, and ranks the first leading position in the world.

Cotton is one of the crops that are most seriously damaged by the pests, and there are at least 300 kinds of cotton pests. Before the cultivation of transgenic insect-resistant (Helicoverpa armigera resistant) cotton, Aphis gossypii, Heliothis armigera, Tetranychus cinnabarinus, and Petinophera gossypiella are the major four pests that are most harmful to the cotton. The annual loss in cotton production due to pests attack is up to 15-20%, and the cost of the pesticide spent for controlling the pests is up to 1200-1800 Yuan/hectare every year. Particularly in the early 1990s, due to the long-term use of chemical pesticides in large amounts, the cotton pests become highly resistant eventually, which directly results in the large outbreak of Helicoverpa armigera in the Yellow River Basin of China, and the damage is uncommon in the history^{[1]}.

With the advancement in biotechnology, the incidence and damage of the Lepidoptera pests including Helicoverpa armigera are fundamentally and effectively controlled by the large area plantation of transgenic Bt cotton. The usage amount of chemical pesticides in the cotton field is reduced considerably, and a series of changes occur consequently to the ecological system of the cotton field, causing the number of plant bugs, a non-target pest, to increase, and the damage thereof to aggravate in the cotton field. Since 2000, the damage from plant bug is constantly aggravated, and the plant bug gradually becomes one of the primary pests from a secondary pest^{[2]}. The control of the plant bug becomes a focus in various regions.

The plant bug belongs to Heteroptera, Miridae, and is a class of notorious pests in cotton production. The common species in China includes Apolygus lucorum (Meyer-Dür), Adelphocoris suturalis (Jakovlev), Adelphocoris fasciaticollis (Reuter), Adelphocoris lineolatus (Goeze) and Lygus pratensis (Linnaeus). The hosts of plant bug include 200 plants from 50 families, such as cotton, fruits, vegetables, alfalfa, tea, and a variety of weeds, including many important agricultural crops^{[3]}. The plant bug preferentially feeds on the flowers, buds, fruits and other reproductive organs of plants, and mainly causes injury to growing points during the seedling stage of the cotton to form headless cotton, and to growing points and young buds during the bud stage of the cotton, which results in damage of the cotton leaves, and the vegetative growth becomes slow with overgrown branches and leaves. Also the cotton plant becomes hollow with the reduction in the bud number, thereby leading to delayed plant maturation, and decline in cotton production. In addition, plant bug has the biological characteristics of omnivorousness, high migration and spread ability, high rate of overlapping generations and high hiding ability which reduce the control effect of pesticides to kill these plant pests.

At present, due to the absence of suitable varieties resistant to plant bug, persistent spraying of chemical insecticides is a vital way to address the insect damage during the growth process of cotton. However, numerous insects in the cotton field are killed by the chemical insecticides, which is not conducive to the ecological balance in the cotton field, causes environmental pollution, and increases planting cost, such that the comparative advantages of the transgenic Bt insect-resistant cotton are reduced. In recent years, due to the persistently increased chemical prevention and control efforts for plant bug in the field, the resistance becomes notable. It is reported that the plant bug is resistant to organophosphorus, pyrethroid, carbamate and cyclodiene insecticides. Resistance will cause great difficulties to the prevention and control work in the future. Therefore, the cultivation of cotton varieties resistant to plant bug by incorporating a plant bug-resistant gene into cotton through a transgenic technology can reduce the application of pesticides, alleviate the environmental pollution, maintain the ecological balance, and save manpower, material resources and social resources, thereby producing enormous economical benefits.

In 2007, the Cry51Aa1 protein isolated from the Bt strain F14-1 and a coding gene sequence thereof were submitted to NCBI GenBank under Accession No. DQ836184 by Huang Dafang. The Cry51Aa1 protein is a parasporal crystal produced in Bacillus thuringiensis, which has important application prospects in the area of biological control^{[4]}.

### LITERATURES

1. Wu K M, Guo Y Y. The evolution of cotton pest management practices in China[J]. Annu. Rev. Entomol., 2005, 50: 31-52.
2. Wu K, Li W, Feng H, et al. Seasonal abundance of the mirids, Lygus lucorum and Adelphocoris spp. (Hemiptera: Miridae) on Bt cotton in northern China[J]. Crop protection, 2002, 21(10): 997-1002.
3. Lu Y H, Qiu F, Feng H Q, et al. Species composition and seasonal abundance of pestiferous plant bugs (Hemiptera: Miridae) on Bt cotton in China[J]. Crop Protection, 2008, 27(3): 465-472.
4. Huang D F, Zhang J, Song F P, et al. Microbial control and biotechnology research on Bacillus thuringiensis in China[J]. Journal of invertebrate pathology, 2007, 95(3): 175-180.

### SUMMARY

The present invention provides an insect-resistant vegetable protein.

The insect-resistant vegetable protein provided in the present invention is designated as MRP001 protein, and is a protein of A1) or A2) expressed by an artificially synthesized gene:

A1) a protein having an amino acid sequence as shown in SEQ ID No.6; and

A2) a protein having insect resistance derived from A1) by substituting, and/or deleting, and/or adding one or more amino acid residues in the amino acid sequence of the protein of A1).

SEQ ID No.6 is composed of 306 amino acid residues.

The MRP001 protein in A2) may be obtained by synthesizing a coding gene thereof followed by biological expression. The coding gene of the MRP001 protein in A2) may be obtained by deleting the codons of one or more amino acid residues in a DNA sequence of nucleotide positions 896-1816 in SEQ ID No.1, and/or undergoing missense mutation of one or more base pairs.

To solve the above technical problems, the present invention further provides biological materials associated with the MRP001 protein.

The biological materials associated with the MRP001 protein provided in the present invention are at least one of B1) to B10):

B1) a nucleic acid molecule encoding the MRP001 protein;

B2) an expression cassette comprising the nucleic acid molecule of B1);

B3) a recombinant vector comprising the nucleic acid molecule of B1), or a recombinant vector comprising the expression cassette of B2);

B4) a recombinant microorganism comprising the nucleic acid molecule of B1), or a recombinant microorganism comprising the expression cassette of B2), or a recombinant microorganism comprising the recombinant vector of B3);

B5) a transgenic plant cell line comprising the nucleic acid molecule of B1), or a transgenic plant cell line comprising the expression cassette of B2), or a transgenic plant cell line comprising the recombinant vector of B3);

B6) a transgenic plant tissue comprising the nucleic acid molecule of B1), or a transgenic plant tissue comprising the expression cassette of B2), or a transgenic plant tissue comprising the recombinant vector of B3);

B7) a transgenic plant organ comprising the nucleic acid molecule of B1), or a transgenic plant organ comprising the expression cassette of B2), or a transgenic plant organ comprising the recombinant vector of B3);

B8) a transgenic plant comprising the nucleic acid molecule of B1), or a transgenic plant comprising the expression cassette of B2), or a transgenic plant comprising the recombinant vector of B3);

B9) a tissue culture produced from regenerable cells of the transgenic plant of B8); and

B10) a protoplast produced from the tissue culture of B9).

In the biological materials associated with the MRP001 protein, the nucleic acid molecule of B1) is a nucleic acid molecule of B1a), B1b), B1c), or B1d):

B1a) a DNA or cDNA molecule having a coding sequence of positions 896 to 1816 of SEQ ID No. 1;

B1b) a cDNA or genomic DNA molecule that is 75% or more identical to the nucleotide sequence defined in B1a) and encodes the MRP001 protein;

B1c) a cDNA or genomic DNA molecule that hybridizes to the nucleotide sequence defined in B1a) under stringent conditions and encodes the MRP001 protein; or

B1d) a DNA molecule that is reversely complementary to the DNA molecule of B1a), B1b), or B1c).

The nucleotide sequence of the nucleic acid molecule encoding the MRP001 protein according to the present invention may be mutated easily by those skilled in the art by using following known methods, for example, directed evolution and point mutation. Those that are artificially modified are 75% or more identical to the nucleotide sequence of the nucleic acid molecule isolated in the present invention and encoding the MRP001 protein, and encode the MRP001 protein, are all derived from and equivalent to the nucleotide sequence of the present invention.

The term “identity” as used herein refers to the sequence similarity to a natural nucleic acid sequence. “Identity” includes a nucleotide sequence that is 75% or more, 85% or more, 90% or more, or 95% or more identical to a DNA or cDNA molecule of the nucleotide positions 896 to 1816 in SEQ ID No.1 of the present invention. The identity may be visibly evaluated or evaluated by computer software. When the computer software is used, the identity between two or more sequences may be expressed in percentages (%), by which the identity between relevant sequences may be evaluated.

The stringent conditions are hybridization in 2×SSC, 0.1% SDS solution at 68° C. and rinsing twice each for 5 min, followed by hybridization in 0.5×SSC, 0.1% SDS solution at 68° C. and rinsing twice each for 15 min.

75% or more identity may be 80%, 85%, 90%, 95% or more identity.

SEQ ID No.1 is composed of 2093 nucleotides, in which the coding sequence is positions 896 to 1816, which encodes the protein as shown in SEQ ID No.6.

Among the biological materials associated with the MRP001 protein, the expression cassette refers to a DNA able to express a corresponding protein in a host cell, and comprising both a promoter initiating the transcription of a relevant gene and also a terminator terminating the transcription of the relevant gene. For example, the expression cassette of B2) comprising the nucleic acid molecule encoding the MRP001 protein refers to a DNA able to express the MRP001 protein in a host cell. Further, the expression cassette may also comprise an enhancer sequence. The promoter useful in the present invention includes, but is not limited to, a constitutive promoter, a tissue, organ or development-specific promoter, and an inducible promoter. The example of promoter includes, but is not limited to, constitutive promoter T7 lac, constitutive cauliflower mosaic virus (CaMV) 35S promoter, ribulose-1,5-bisphospate carboxylase (rbcs) small subunit gene promoter; wound inducible promoter, leucine aminopeptidase (“LAP”, Chao et al. (1999) Plant Physiol. 120:979-992), from tomato; chemically inducible promoter pathogenesis-related protein 1 (PR1) gene promoter from tobacco (which is induced by salicylic acid and BTH (benzthiadiazole-7-thiocarboxylate S-methyl)); tomato proteinase inhibitor II promoter (PIN2) or LAP promoter (both of which can be induced by methyl jasmonate); a heat shock promoter (U.S. Pat. No. 5,187,267); a tetracycline inducible promoter (U.S. Pat. No. 5,057,422); a seed specific promoter, for example, millet seed specific promoter pF128 (CN101063139B (Chinese Patent No. 200710099169.7)), and a seed storage protein specific promoter (e.g., phaseolin, napin, oleosin, and soybean beta conglycin promoters (Beachy et al. (1985) EMBO J. 4:3047-3053)). All the literatures cited here are incorporated by reference in their entirety. The suitable transcription terminator includes, but is not limited to, T7 terminator, Agrobacterium tumefaciens nopaline synthetase terminator (NOS terminator), cauliflower mosaic virus (CaMV) 35S terminator, tml terminator, pea rbcS E9 terminator, and nopaline and octopine synthetase terminators (see, for example, Odell et al. (1985), Nature, 313:810; Rosenberg et al. (1987), Gene, 56:125; Guerineau et al. (1991), Mol. Gen. Genet, 262:141; Proudfoot (1991), Cell, 64:671; Sanfacon et al., Genes Dev., 5:141; Mogen et al. (1990), Plant Cell, 2:1261; Munroe et al. (1990), Gene, 91:151; Ballad et al. (1989), Nucleic Acids Res. 17:7891; and Joshi et al. (1987), Nucleic Acid Res., 15:9627). In an embodiment of the present invention, the promoter initiating the transcription of the MRP001 gene in the MRP001 gene expression cassette in E. Coli BL21 (DE3) recipient cell may be the constitutive promoter T7 lac, and the terminator terminating the transcription of the MRP001 gene may be the T7 terminator. The promoter initiating the transcription of the MRP001 gene in the MRP001 gene expression cassette in the recipient plant cotton is the constitutive cauliflower mosaic virus (CaMV) 35S promoter, which may specifically have a DNA sequence as shown in positions 1 to 860 of SEQ ID No.1; and the terminator terminating the transcription of the MRP001 gene may be the NOS terminator, which may specifically have a DNA sequence as shown in positions 1830 to 2093 of SEQ ID No.1.

Among the biological materials associated with the MRP001 protein, the recombinant vector containing the MRP001 gene expression cassette may be constructed with an existing prokaryotic vector. Among the biological materials associated with the MRP001 protein, the vector may be a plasmid, a cosmid, a phage or a viral vector. Among the biological materials associated with the MRP001 protein, the recombinant vector of B3) may comprise a DNA sequence for encoding the MRP001 protein as shown in positions 896 to 1816 of SEQ ID No.1. Further, the recombinant vector of B3) may be specifically pET28a-MRP001, in which the DNA sequence between the NedI and XhoI recognition sites (recognition sequences) in the vector pET28a is replaced by the DNA sequence for encoding the MRP001 protein as shown in positions 896 to 1816 of SEQ ID No.1, and the remaining DNA sequences are kept unchanged, to obtain the recombinant vector expressing the MRP001 protein of SEQ ID No. 6.

The recombinant vector containing the MRP001 gene expression cassette may be constructed with an existing plant expression vector, for example, pCAMBIA2301, pET-28a, pSP72, pROKII, pBin438, pCAMBIA1302, pCAMBIA1301, pCAMBIA1300, pBI121, pCAMBIA1391-Xa, or pCAMBIA1391-Xb (CAMBIA Corporation). The MRP001 gene vector may further comprise a 3′-terminal non-translated region of a foreign gene, that is, a polyadenylation signal and any other DNA fragments involved in mRNA processing or gene expression. The polyadenylation signal may direct the addition of p(A) to the 3′ terminus of a mRNA precursor. For example, the non-translated region transcripted at the 3′ terminus of the Agrobacterium tumefaciens tumor-inducing (Ti) plasmid gene (e.g. nopaline synthase Nos gene) and vegetable gene (e.g. soybean storage protein gene) both have similar function. When a plant expression vector is constructed with the gene of the present invention, an enhancer may be included, including a translational enhancer or a transcriptional enhancer. These enhancer regions may be the ATG initiation codon or an initiation codon of an adjacent region, which however needs to be co-framed with the coding sequence, to ensure the proper translation of the whole sequence. The translation control signal and the initiation codon are widely available, and may be natural, or synthesized. The translation initiation region may be from a transcription initiation region or a structural gene. For ease of identification and screening of transgenic plant cell or plant, the plant expression vector may be processed, for example, by adding a gene (GUS gene, luciferase gene, and the like) that can be expressed in a plant and encodes an enzyme or a luminescent compound that can produce the color change, an antibiotic marker gene (for example, nptII gene imparting resistance to kanamycin and related antibiotics, bar gene imparting resistance to the herbicide phosphinothricin, hph gene imparting resistance to the antibiotic homomycin, dhfr gene imparting resistance to methatrexate, and EPSPS gene imparting resistance to glyphosate), a chemically labeled gene (for example, herbicide resistant gene), and a mannose-6phosphate isomerase gene providing the mannose metabolizing ability. The recombinant vector of B3) may comprise a DNA sequence encoding the MRP001 protein as shown in positions 896 to 1816 of SEQ ID No.1. Further, the recombinant vector of B3) may specifically be pCambia2301-35S-MRP001-NOS. The pCambia2301-35S-MRP001-NOS is a recombinant vector expressing the MRP001 protein of SEQ ID No.6 obtained by replacing the DNA sequence between the HindIII and EcoRI recognition sites (recognition sequences) of the vector pCambia2301 with the DNA sequence of SEQ ID No.1, and keeping the remaining DNA sequences unchanged.

Among the biological materials associated with the MRP001 protein, the recombinant microorganism of B4) may be specifically a bacterium, a yeast, an algae, and a fungi. The bacterium may be from Escherichia, Erwniia, Agrobacterium, Flavobacterium, Alcaligenes, Pseudomonas, and Bacillus. In an embodiment of the present invention, the microorganism may be specifically Escherichia, and further specifically E. Coli. BL21(DE3).

Among the biological materials associated with the MRP001 protein, the transgenic plant organ of B7) may be the root, stem, leaf, flower, fruit, and seed of a transgenic plant.

Among the biological materials associated with the MRP001 protein, the tissue culture of B9) may be derived from the root, stem, leaf, flower, fruit, seed, pollen, germ, and anther.

The present invention further provides use of any one of 1) to 5):

1) use of the MRP001 protein in the regulation of plant resistance to insects;

2) use of the MRP001 protein in the preparation of insect-resistant plant products;

3) use of the MRP001 protein associated biological materials in the regulation of plant resistance to insects;

4) use of the MRP001 protein associated biological materials in the preparation of insect-resistant plant products; and

5) use of the MRP001 protein associated biological materials in the cultivation of insect-resistant plants.

The present invention also provides a botanical insecticide.

The botanical insecticide provided in the present invention contains the MRP001 protein.

In the botanical insecticide, the botanical insecticide may have the MRP001 protein or a combination of the MRP001 protein with other insecticidal substances as the active ingredient(s).

The present invention further provides a method for preparing the MRP001 protein.

The method for preparing the MRP001 protein provided in the present invention comprises introducing a gene encoding the MRP001 protein according to claim 1 to a recipient cell, to obtain a recombinant cell, and culturing the recombinant cell, to obtain the MRP001 protein.

The recipient cell is a microorganism cell, a non-human animal cell, or a plant cell.

The microorganism cell may be a bacterium or fungi. The bacterium may be a Gram negative bacterium. The Gram negative bacterium may be Escherichia, and further the Escherichia may be E. Coli BL21(DE3).

The protein prepared by the method for preparing the MRP001 protein may be a powder or an aqueous solution.

The present invention further provides a method for cultivating an insect-resistant transgenic plant.

The method for cultivating an insect-resistant transgenic plant provided in the present invention comprises the step of introducing a gene encoding the MRP001 protein to a recipient plant, to obtain an insect-resistant transgenic plant.

Hereinbefore, the gene encoding the MRP001 protein is a nucleic acid molecule of B1a), B1b), B1c), or B1d):

B1a) a DNA or cDNA molecule having a coding sequence as shown in positions 896 to 1816 of SEQ ID No. 1;

B1b) a cDNA or genomic DNA molecule that is 75% or more identical to the nucleotide sequence defined in B1a) and encodes the MRP001 protein;

B1c) a cDNA or genomic DNA molecule that hybridizes to the nucleotide sequence defined in B1a) under stringent conditions and encodes the MRP001 protein; or

B1d) a DNA molecule that is reversely complementary to the DNA molecule of B1a), B1b), or B1c).

The gene encoding the MRP001 protein may be modified as follows before being introduced into the recipient cotton, to achieve a better expression effect.

1) Modification and optimization as practically desired, to efficiently express the gene. For example, based on the codon preferred by the recipient plant, the codon is changed while the amino acid sequence of the gene encoding the MRP001 protein according to the present invention is maintained, to conform with plant preferences. During the optimization process, a certain GC content is preferably maintained in the coding sequence after optimization, to best achieve the high expression of the introduced gene in the plant, where the GC content may be 35%, more than 45%, more than 50%, or more than about 60%.

2) Modification of a gene sequence adjacent to the initiating methionine, to effectively initiate the translation. For example, the modification is carried out with a sequence known to be effective in a plant.

3) Linking to a promoter expressed in various plants, to facilitate the expression in plants. The promoter may include a constitutive, inducible, temporally regulated, developmentally regulated, chemically regulated, tissue preferred, and tissue specific promoter. The choice of promoter will vary depending on the temporal and spatial requirements for expression, and also depending on the target species. For example, the choice of a tissue- or organ-specific expression promoter depends on the stage of development of the required recipient.

4) Linking to a suitable transcription terminator, which can also increase the expression efficiency of the gene of the present invention. For example, the CaMV-derived tml, the rbcS-derived E9, and any terminators available that are known to function in plants may be linked to the gene of the present invention.

5) Introduction of an enhancer sequence, for example, intron sequences (e.g. from Adh1 and bronzei) and viral leader sequences (e.g. from TMV, MCMV and AMV).

In the above method, the MRP001 gene is incorporated into the recipient plant by a recombinant expression vector (MRP001 gene expression vector) containing the MRP001 gene expression cassette, where in the MRP001 gene expression cassette, the promoter initiating the transcription of the MRP001 gene is cauliflower mosaic virus 35S promoter.

The MRP001 gene expression vector may be incorporated into a plant cell or tissue by a Ti plasmid, a plant virus vector, direct DNA transformation, microinjection, electroporation, Agrobacterium-mediated transformation, and other conventional biotechnical methods.

The method further comprises the step of screening a plant expressing the coding gene out of the plants incorporated with the gene encoding the MRP001 protein as shown in positions 896 to 1816 of SEQ ID No.1, to obtain the transgenic cotton.

Hereinbefore, it should be understood that the transgenic plant comprises not only the first generation of transgenic plant obtained by transforming the recipient plant with the gene, but also its progenies. For the transgenic plant, the gene may be proliferated in the species, or transferred to other varieties, especially commercial varieties, of the same species, by a conventional breeding technology. The transgenic plant includes seeds, callus, whole plant, and cells.

In the use, botanical insecticide, or method, the plant may be a host of plant bug, which may be a dicotyledonous plant for example cotton, apple, tea, alfalfa and so on; or a monocotyledonous plant, for example, rice. The host of the plant bug may be specifically cotton.

Hereinbefore, the insect is plant bug.

It is confirmed through experiments that the recombinant E. Coli BL21-MRP001 constructed by incorporating the MRP001 gene of the present invention into E. Coli BL21(DE3) competent cells can express the MRP001 protein. The protein has a good plant bug killing effect. The mortality of plant bug can be up to 96.7% when the protein concentration is 25 μg/mL, which is far greater than the plant bug mortality in the blank control group and the treatment groups with the same concentration of other four proteins (MRP002 protein, MRP003 protein, MRP004 protein, and MRP005 protein). The transgenic cotton obtained by transferring the MRP001 gene of the present to the cotton has a high resistance to plant bug compared with the cotton in control group 1 and control group 2. The leaf injury index of the MRP001 transgenic cotton is 0.35, and the decline in leaf injury index is 84.75%. The square injured rate is 2.89%, and the decline square injured rate is 88.89%. The present invention is useful in the preparation of biological anti-insect agent containing the protein, and in the cultivation of insect-resistant transgenic cotton, fruits, tea, rice, vegetables and other crops, thereby reducing the application of pesticides and alleviating the environmental pollution, and thus being of great economic value and broad application prospect.

### BRIEF DESCRIPTION OF THE DRAWINGS

FIG. 1 is a cleavage map of the recombinant vector pET28a-MRP001, in which Lane 1 is a band of the recombinant vector pET28a-MRP001 after cleavage; and Lane 2 is a band of a DNA Marker.

FIG. 2 is a schematic structural diagram of the plant expression vector pCambia2301-35S-MRP001-NOS.

FIG. 3 is a cleavage map of the plant expression vector pCambia2301-35S-MRP001-NOS, in which Lane 1 is a band of the plant expression vector pCambia2301-35S-MRP001-NOS after cleavage; and Lane 2 is a band of a DNA Marker.

FIG. 4 is an electropherogram of detecting the MRP001 gene inserted in a transgenic cotton by PCR, in which Lanes 1-5 are DNA samples of the transgenic cotton, Lane n is water (negative control), Lane p is pCambia2301-35S-MRP001-NOS plasmid (positive control), and Lane m is a DNA Marker.

FIG. 5 shows various growth stages of cotton transformed with the MRP001 gene by Agrobacterium-mediated transformation, in which a is a hypocotyl section, b is a callus, c is an embryonic callus, d is an embryoid, e is a regrown seedling, and f is a transgenic plant.

### DETAILED DESCRIPTION

Hereinafter, the present invention is described in further detail with reference to specific examples, which are provided merely for illustrating, instead of limiting the scope of the present invention.

The methods given in examples below are all conventional methods, unless it is otherwise stated.

The materials and reagents used in examples below are all commercially available, unless it is otherwise stated.

The CCRI 24 mentioned in the examples below (Wang Xinyong, and Liu Yining. CCRI 24 performance and cultivation techniques in North Xinjiang [J]. China Cotton, 1998, 25(1):31-31.) may be publicly available from National Cotton Germplasm Medium-term Genebank, Cotton Research Institute of Chinese Academy of Agricultural Sciences. The biological material is merely used for repeating relevant experiments in the present invention, and not for other purposes.

The prokaryotic expression vector pET28a in the examples below is a product available from Novagen, Inc (catalog#69864-3).

The BL21(DE3) competent cell in the examples below is a product available from Beijing TransGen Biotech Co., Ltd (catalog#CD601-01).

The expression vector pCambia2301 in the examples below is a product available from Beijing Dingguo Changsheng Biotech Co. Ltd (catalog#MCV037).

The Agrobacterium LBA4404 in the examples below is a product available from Beijing Dingguo Changsheng Biotech Co. Ltd (catalog#MCCO26).

### Example 1. Preparation and Biological Assay of Protein MRP001 Resistant to Plant Bug

The full-length genes MRP001, MRP002, MRP003, MRP004 and MRP005 were synthesized by Shanghai Bioengineering Co., Ltd.

I. Preparation of Recombinant Bacterial Cell Expressing MRP001 Protein

Recombinant bacterial cell expressing the MRP001 protein was constructed by incorporating a DNA encoding the MRP001 protein to a recipient cell, to obtain a recombinant bacterial cell expressing the MRP001 protein. The DNA encoding the MRP001 protein had a nucleotide sequence as shown in positions 896 to 1816 of SEQ ID No.1 in the sequencing list. The method was specifically as follows.

The DNA sequence between the NdeI and XhoI recognition sites (recognition sequences) in the expression vector pET-28a was replaced by the DNA sequence encoding MRP001 as shown in positions 896 to 1816 of SEQ ID No.1, and keeping the remaining DNA sequences unchanged, to obtain a recombinant vector pET28a-MRP001.

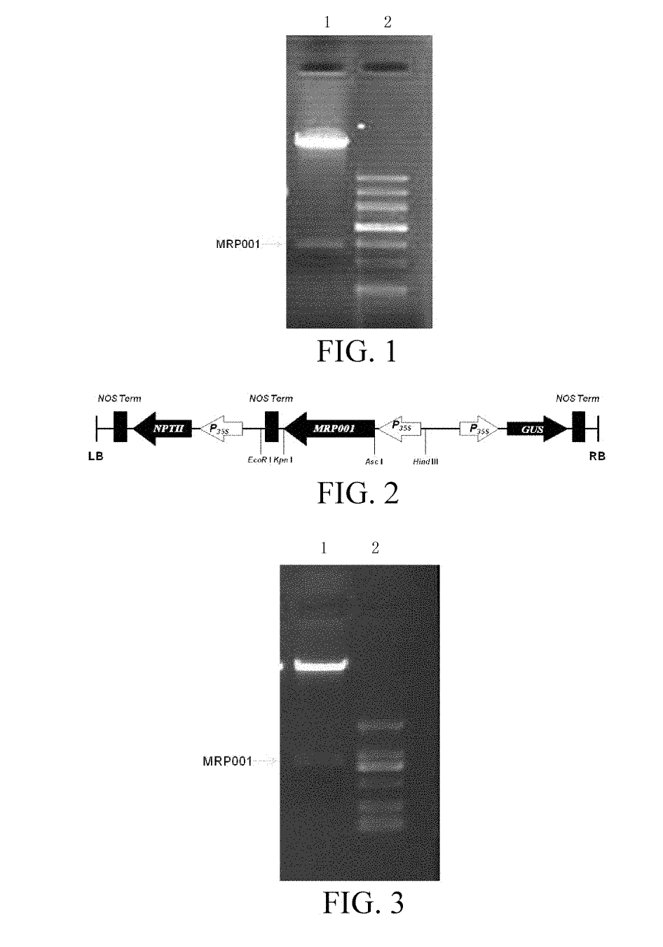

It was confirmed by identification through cleavage (FIG. 1) and DNA sequencing that the nucleotide sequence of the MRP001 gene in pET28a-MRP001 was the nucleotide sequence as shown in positions 896 to 1816 of SEQ ID No.1. The pET28a-MRP001 could express the MRP001 protein of SEQ ID No.6, and the sequence of positions 896 to 1816 of SEQ ID No.1 was the MRP001 encoding sequence.

The DNA sequence between the NdeI and XhoI restriction sites of the recombinant vector pET28a-MRP001 was replaced by the DNA sequence of SEQ ID No.2, and the remaining DNA sequences were kept unchanged, to obtain a recombinant vector pET28a-MRP002 expressing the MRP002 protein. The DNA sequence between the NdeI and XhoI restriction sites of the recombinant vector pET28a-MRP001 was replaced by the DNA sequence of SEQ ID No.3, and the remaining DNA sequences were kept unchanged, to obtain a recombinant vector pET28a-MRP003 expressing the MRP003 protein. The DNA sequence between the NdeI and XhoI restriction sites of the recombinant vector pET28a-MRP001 was replaced by the DNA sequence of SEQ ID No.4, and the remaining DNA sequences were kept unchanged, to obtain a recombinant vector pET28a-MRP004 expressing the MRP004 protein. The DNA sequence between the NdeI and XhoI restriction sites of recombinant vector pET28a-MRP001 was replaced by the DNA sequence of SEQ ID No.5, and the remaining DNA sequences were kept unchanged, to obtain a recombinant vector pET28a-MRP005 expressing the MRP005 protein.

The pET28a-MRP001 was incorporated into E. Coli BL21(DE3) competent cells, to obtain a recombinant strain containing the gene encoding the MRP001 protein as shown in positions 896 to 1816 of SEQ ID No.1, which was designated as recombinant E. Coli BL21-MRP001 (referred to as BL21-MRP001 strain hereinafter). The pET28a-MRP002 was incorporated into E. Coli BL21(DE3) competent cells, to obtain a recombinant strain containing the gene encoding the MRP002 protein as shown in SEQ ID No.2, which was designated as recombinant E. Coli BL21-MRP002 (referred to as BL21-MRP002 strain hereinafter). The pET28a-MRP003 was incorporated to E. Coli BL21(DE3) competent cells, to obtain a recombinant strain containing the gene encoding the MRP003 protein as shown in SEQ ID No.3, which was designated as recombinant E. Coli BL21-MRP003 (referred to as BL21-MRP003 strain). The pET28a-MRP004 was incorporated into E. Coli BL21(DE3) competent cells, to obtain a recombinant strain containing the gene encoding the MRP004 protein as shown in SEQ ID No.4, which was designated as recombinant E. Coli BL21-MRP004 (referred to as BL21-MRP004 strain hereinafter). The pET28a-MRP005 was incorporated into E. Coli BL21(DE3) competent cells, to obtain a recombinant strain containing the gene encoding the MRP005 protein as shown in SEQ ID No.5, which was designated as recombinant E. Coli BL21-MRP005 (referred to as BL21-MRP005 strain hereinafter). The expression vector pET28a was incorporated into E. Coli BL21(DE3) competent cells, to obtain a recombinant strain containing no insert fragment, which was designated as recombinant E. Coli BL21-CK (referred to as BL21-CK strain hereinafter), and was an empty vector transformed control strain.

II. Expression and Extraction of MRP001 Protein in and from Recombinant Strain

1. Collection of Bacterial Cells from Various Treatments

The BL21-MRP001 strain, BL21-MRP002 strain, BL21-MRP003 strain, BL21-MRP004 strain, BL21-MRP005 strain, and BL21-CK strain obtained in Step I were respectively inoculated in a test tube containing 5 mL of an LB liquid medium (in which the content of kanamycin is 50 mg/mL), and the BL21 strain was inoculated in a kanamycin-free LB liquid medium, and incubated overnight at 37° C. in a shaker, to obtain a seed culture of the BL21-MRP001 strain, BL21-MRP002 strain, BL21-MRP003 strain, BL21-MRP004 strain, BL21-MRP005 strain, BL21-CK strain, and BL21 strain respectively. The seed culture above was inoculated respectively in a test tube containing 5 mL of an LB liquid medium in an amount of 1% by volume, and incubated overnight at 37° C. in a shaker until the OD₆₀₀ value reached 0.4-1. 5 mL of 1 mM IPTG solution was added respectively to the bacterial culture having an OD₆₀₀ value of 0.4-1, continuously incubated at 37° C. for another 4 hrs in a shaker, and centrifuged at 4000 rpm for 10 min to collect the bacterial pellet. In this way, the bacteria of the IPTG induced BL21-MRP001 strain, BL21-MRP002 strain, BL21-MRP003 strain, BL21-MRP004 strain, BL21-MRP005 strain, BL21-CK strain, and BL21 strain were obtained.

2. Protein Extraction from Recombinant Strain

36 mL of a lysis buffer (containing 2 mM Tris-HCl, and 0.2 mM CaCl₂, pH 8.0) was added respectively to the bacteria of the IPTG induced BL21-MRP001 strain, BL21-MRP002 strain, BL21-MRP003 strain, BL21-MRP004 strain, BL21-MRP005 strain, BL21-CK strain, and BL21 strain, to re-suspend the bacteria. 4 mL of 10 mg/mL lysozyme was added respectively to the resuspended bacterial solution, to obtain a resuspended bacterial solution containing 1 mg/mL of lysozyme, which was then stood on ice for 30 min. After standing in the ice bath, the bacteria were homogenized by ultrasonication (at a power of 400 w for a duration of 20 min in total, where the ultrasonication was continued for is at each time and cyclically carried out at an interval of 2 s). Following ultrasonication, the solution was centrifuged at 4000 rpm for 10 min, to collect a supernatant. In this way, the supernatant of the BL21-MRP001 strain, the supernatant of the BL21-MRP002 strain, the supernatant of the BL21-MRP003 strain, the supernatant of the BL21-MRP004 strain, the supernatant of the BL21-MRP005 strain, the supernatant of the BL21-CK strain, and the supernatant of the BL21 strain were obtained respectively.

3. Protein Quantification

The protein expressed in the BL21-MRP001 strain was designated as MRP001 protein, the protein expressed in the BL21-MRP002 strain was designated as MRP002 protein, the protein expressed in the BL21-MRP003 strain was designated as MRP003 protein, the protein expressed in the BL21-MRP004 strain was designated as MRP004 protein, the protein expressed in the BL21-MRP005 strain was designated as MRP005 protein, the protein expressed in the BL21-CK strain was designated as BL21-CK protein, and the protein expressed in the BL21 strain was designated as BL21 protein.

The protein in the supernatant of the BL21-MRP001 strain, BL21-MRP002 strain, BL21-MRP003 strain, BL21-MRP004 strain, BL21-MRP005 strain, BL21-CK strain and BL21 strain was quantified by using SK3071 Non-Interfering Protein Concentration Determination Kit available from Shanghai Bioengineering Co., Ltd. The results are as follows. The MRP001 protein concentration in the supernatant of the BL21-MRP001 strain is 0.37 mg/mL, the MRP002 protein concentration in the supernatant of the BL21-MRP002 strain is 0.58 mg/mL, the MRP003 protein concentration in the supernatant of the BL21-MRP003 strain supernatant is 0.1 mg/mL, the MRP004 protein concentration in the supernatant of the BL21-MRP004 strain is 1.33 mg/mL, the MRP005 protein concentration in the supernatant of the BL21-MRP005 strain is 1.47 mg/mL, the BL21-CK protein concentration in the supernatant of the BL21-CK strain is 1.16 mg/mL, and the BL21 protein concentration in the supernatant of the BL21 strain is 0.3 mg/mL. The MRP001 protein in the supernatant of the BL21-MRP001 strain, the MRP002 protein in the supernatant of the BL21-MRP002 strain, the MRP003 protein in the supernatant of the BL21-MRP003 strain, the MRP004 protein in the supernatant of the BL21-MRP004 strain, the MRP005 protein in the supernatant of the BL21-MRP005 strain, the BL21-CK protein in the supernatant of the BL21-CK strain, and the BL21 protein in the supernatant of the BL21 strain were freeze-dried into powders, to obtain MRP001 protein powder, MRP002 protein powder, MRP003 protein powder, MRP004 protein powder, MRP005 protein powder, BL21-CK protein powder, and BL21 protein powder respectively, which were stored at −80° C. for later use.

III. Bioassay of MRP001 Protein

The composition of the conventional liquid feed was: sucrose 2.8 g, beer yeast powder 0.25 g, 50% honey water 2.5 g, egg 22.5 g, wheat germ 10 g, lima bean flour 30 g, soybean flour 2.5 g, yolk 30 g, soybean lecithin 1.5 g, compound vitamin 1.2 g, and water 164 g.

The MRP001 protein powder obtained in Step II was added to the conventional liquid feed, to obtain a 12.5 μg/mL MRP001 protein liquid feed (in which the concentration of the MRP001 protein in the liquid feed was 12.5 μg/mL), a 25 μg/mL MRP001 protein liquid feed (in which the concentration of the MRP001 protein in the liquid feed was 25 μg/mL), a 50 μg/mL MRP001 protein liquid feed (in which the concentration of the MRP001 protein in the liquid feed was 50 μg/mL), a 100 μg/mL MRP001 protein liquid feed (in which the concentration of the MRP001 protein in the liquid feed was 100 μg/mL), and a 200 μg/mL MRP001 protein liquid feed (in which the concentration of the MRP001 protein in the liquid feed was 200 μg/mL). The MRP002 protein powder obtained in Step II was added to the conventional liquid feed, to obtain a 12.5 μg/mL MRP002 protein liquid feed (in which the concentration of the MRP002 protein in the liquid feed was 12.5 μg/mL), a 25 μg/mL MRP002 protein liquid feed (in which the concentration of the MRP002 protein in the liquid feed was 25 μg/mL), a 50 μg/mL MRP002 protein liquid feed (in which the concentration of the MRP002 protein in the liquid feed was 50 μg/mL), a 100 μg/mL MRP002 protein liquid feed (in which the concentration of the MRP002 protein in the liquid feed was 100 μg/mL) and a 200 μg/mL MRP002 protein liquid feed (in which the concentration of the MRP002 protein in the liquid feed was 200 μg/mL). The MRP003 protein powder obtained in Step II was added to the conventional liquid feed, to obtain a 12.5 μg/mL MRP003 protein liquid feed (in which the concentration of the MRP003 protein in the liquid feed was 12.5 μg/mL), a 25 μg/mL MRP003 protein liquid feed (in which the concentration of the MRP003 protein in the liquid feed was 25 μg/mL), a 50 μg/mL MRP003 protein liquid feed (in which the concentration of the MRP003 protein in the liquid feed was 50 μg/mL), a 100 μg/mL MRP003 protein liquid feed (in which the concentration of the MRP003 protein in the liquid feed was 100 μg/mL), and a 200 μg/mL MRP003 protein liquid feed (in which the concentration of the MRP003 protein in the liquid feed was 200 μg/mL). The MRP004 protein powder obtained in Step II was added to the conventional liquid feed, to obtain a 12.5 μg/mL MRP004 protein liquid feed (in which the concentration of the MRP004 protein in the liquid feed was 12.5 μg/mL), a 25 μg/mL MRP004 protein liquid feed (in which the concentration of the MRP004 protein in the liquid feed was 25 μg/mL), a 50 μg/mL MRP004 protein liquid feed (in which the concentration of the MRP004 protein in the liquid feed was 50 μg/mL), a 100 μg/mL MRP004 protein liquid feed (in which the concentration of the MRP004 protein in the liquid feed was 100 μg/mL), and a 200 μg/mL MRP004 protein liquid feed (in which the concentration of the MRP004 protein in the liquid feed was 200 μg/mL). The MRP005 protein powder obtained in Step II was added to the conventional liquid feed, to obtain a 12.5 μg/mL MRP005 protein liquid feed (in which the concentration of the MRP005 protein in the liquid feed was 12.5 μg/mL), a 25 μg/mL MRP005 protein liquid feed (in which the concentration of the MRP005 protein in the liquid feed was 25 μg/mL), a 50 μg/mL MRP005 protein liquid feed (in which the concentration of the MRP005 protein in the liquid feed was 50 μg/mL), a 100 μg/mL MRP005 protein liquid feed (in which the concentration of the MRP005 protein in the liquid feed was 100 μg/mL) and a 200 μg/mL MRP005 protein liquid feed (in which the concentration of the MRP005 protein in the liquid feed was 200 μg/mL). The control group 1 was the conventional liquid feed. The control group 2 was a 200 μg/mL BL21-CK protein liquid feed (in which the concentration of the BL21-CK protein in the liquid feed was 200 μg/mL) obtained by adding the BL21-CK protein powder obtained in Step II to the conventional liquid feed. The control group 3 was a 200 μg/mL BL21 protein liquid feed (in which the concentration of the BL21 protein in the liquid feed was 200 μg/mL) obtained by adding the BL21 protein powder obtained in Step II to the conventional liquid feed. The parafilm was cut into squares of 2 cm×2 cm. Then 500 μL of the above concentrations of protein liquid feeds were positioned in and enwrapped by a stretched parafilm, and slightly squeezed to have spherical shape, for ease of sucking by Apolygus lucorum.

Any one of the above concentrations of protein liquid feeds enwrapped with a parafilm to have a spherical shape was placed in each test tube, and the weight of the protein liquid feed in each test tube was the same. Then 20 newly hatched and unfed Apolygus lucorum were inoculated and cultured in an environment having a temperature of 26-28° C., and a relative humidity of 70%. The mortality was statistically calculated at days 3, 6, and 9 after inoculation. The experiment was repeated 3 times, and each feed was used for test in 3 tubes in each repetition. The mortality is calculated by a formula: Mortality=number of dead bugs/total number of inoculated bugs×100%.

The treatment group in which the Apolygus lucorum was fed with the 12.5 μg/mL MRP001 protein liquid feed was designated as 12.5 μg/mL BL21-MRP001 group. The treatment group in which the Apolygus lucorum was fed with the 25 μg/mL MRP001 protein liquid feed was designated as 25 μg/mL BL21-MRP001 group. The treatment group in which the Apolygus lucorum was fed with the 50 μg/mL MRP001 protein liquid feed was designated as 50 μg/mL BL21-MRP001 group. The treatment group in which the Apolygus lucorum was fed with the 100 μg/mL MRP001 protein liquid feed was designated as 100 μg/mL BL21-MRP001 group. The treatment group in which the Apolygus lucorum was fed with the 200 μg/mL MRP001 protein liquid feed was designated as 200 μg/mL BL21-MRP001 group. The treatment group in which the Apolygus lucorum was fed with the 12.5 μg/mL MRP002 protein liquid feed was designated as 12.5 μg/mL BL21-MRP002 group. The treatment group in which the Apolygus lucorum was fed with the 25 μg/mL MRP002 protein liquid feed was designated as 25 μg/mL BL21-MRP002 group. The treatment group in which the Apolygus lucorum was fed with the 50 μg/mL MRP002 protein liquid feed was designated as 50 μg/mL BL21-MRP002 group. The treatment group in which the Apolygus lucorum was fed with the 100 μg/mL MRP002 protein liquid feed was designated as 100 μg/mL BL21-MRP002 group. The treatment group in which the Apolygus lucorum was fed with the 200 μg/mL MRP002 protein liquid feed was designated as 200 μg/mL BL21-MRP002 group. The treatment group in which the Apolygus lucorum was fed with the 12.5 μg/mL MRP003 protein liquid feed was designated as 12.5 μg/mL BL21-MRP003 group. The treatment group in which the Apolygus lucorum was fed with the 25 μg/mL MRP003 protein liquid feed was designated as 25 μg/mL BL21-MRP003 group. The treatment group in which the Apolygus lucorum was fed with the 50 μg/mL MRP003 protein liquid feed was designated as 50 μg/mL BL21-MRP003 group. The treatment group in which the Apolygus lucorum was fed with the 100 μg/mL MRP003 protein liquid feed was designated as 100 μg/mL BL21-MRP003 group. The treatment group in which the Apolygus lucorum was fed with the 200 μg/mL MRP003 protein liquid feed was designated as 200 μg/mL BL21-MRP003 group. The treatment group in which the Apolygus lucorum was fed with the 12.5 μg/mL MRP004 protein liquid feed was designated as 12.5 μg/mL BL21-MRP004 group. The treatment group in which the Apolygus lucorum was fed with the 25 μg/mL MRP004 protein liquid feed was designated as 25 μg/mL BL21-MRP004 group. The treatment group in which the Apolygus lucorum was fed with the 50 μg/mL MRP004 protein liquid feed was designated as 50 μg/mL BL21-MRP004 group. The treatment group in which the Apolygus lucorum was fed with the 100 μg/mL MRP004 protein liquid feed was designated as 100 μg/mL BL21-MRP004 group. The treatment group in which the Apolygus lucorum was fed with the 200 μg/mL MRP004 protein liquid feed was designated as 200 μg/mL BL21-MRP004 group. The treatment group in which the Apolygus lucorum was fed with the 12.5 μg/mL MRP005 protein liquid feed was designated as 12.5 μg/mL BL21-MRP005 group. The treatment group in which the Apolygus lucorum was fed with the 25 μg/mL MRP005 protein liquid feed was designated as 25 μg/mL BL21-MRP005 group. The treatment group in which the Apolygus lucorum was fed with the 50 μg/mL MRP005 protein liquid feed was designated as 50 μg/mL BL21-MRP005 group. The treatment group in which the Apolygus lucorum was fed with the 100 μg/mL MRP005 protein liquid feed was designated as 100 μg/mL BL21-MRP005 group. The treatment group in which the Apolygus lucorum was fed with the 200 μg/mL MRP005 protein liquid feed was designated as 200 μg/mL BL21-MRP005 group. The treatment group in which the Apolygus lucorum was fed with the conventional liquid feed was designated as control group 1. The treatment group in which the Apolygus lucorum was fed with the 200 μg/mL BL21-CK protein liquid feed was designated as control group 2. The treatment group in which the Apolygus lucorum was fed with the 200 μg/mL BL21 protein liquid feed was designated as control group 3.

The results are as shown in Table 1. The artificially synthesized MRP001 protein has a good Apolygus lucorum killing effect. The mortality of Apolygus lucorum can be up to 96.7% when the protein concentration is 25 μg/mL, which is far greater than the Apolygus lucorum mortality in the control group and the treatment groups with the same concentration of other four proteins.

TABLE 1
Results from bioassay of various proteins on Apolygus lucorum

| | Mortality | | |
|---|---|---|---|
| Treatment group | Day 3 | Day 6 | Day 9 |
| Control group 1 | 0.167 ± 0.076376 | 0.283 ± 0.076376 | 0.283 ± 0.076376 |
| Control group 2 | 0.100 ± 1.69967E−17 | 0.233 ± 0.076376262 | 0.267 ± 0.1040833 |
| Control group 3 | 0.067 ± 0.028868 | 0.183 ± 0.104 | 0.200 ± 0.132 |
| 12.5 μg/mL BL21-MRP001 | 0.117 ± 0.028868 | 0.167 ± 0.028868 | 0.183 ± 0.028868 |
| group | | | |
| 25 μg/mL BL21-MRP001 | 0.966667 ± 0.057735 | 0.966667 ± 0.057735 | 0.966667 ± 0.057735 |
| group | | | |
| 50 μg/mL BL21-MRP001 | 1.000 ± 0.000 | 1.000 ± 0.000 | 1.000 ± 0.000 |
| group | | | |
| 100 μg/mL BL21-MRP001 | 1.000 ± 0.000 | 1.000 ± 0.000 | 1.000 ± 0.000 |
| group | | | |
| 200 μg/mL BL21-MRP001 | 1.000 ± 0.000 | 1.000 ± 0.000 | 1.000 ± 0.000 |
| group | | | |
| 12.5 μg/mL BL21-MRP002 | 0.083 ± 0.076376 | 0.117 ± 0.104083 | 0.150 ± 0.1 |
| group | | | |
| 25 μg/mL BL21-MRP002 | 0.017 ± 0.028868 | 0.067 ± 0.028868 | 0.167 ± 0.028868 |
| group | | | |
| 50 μg/mL BL21-MRP002 | 0.417 ± 0.076376 | 0.583 ± 0.160728 | 0.600 ± 0.132288 |
| group | | | |
| 100 μg/mL BL21-MRP002 | 1.000 ± 0.000 | 1.000 ± 0.000 | 1.000 ± 0.000 |
| group | | | |
| 200 μg/mL BL21-MRP002 | 1.000 ± 0.000 | 1.000 ± 0.000 | 1.000 ± 0.000 |
| group | | | |
| 12.5 μg/mL BL21-MRP003 | 0.200 ± 0.217945 | 0.233 ± 0.189297 | 0.233 ± 0.189297 |
| group | | | |
| 25 μg/mL BL21-MRP003 | 0.133 ± 0.076376 | 0.200 ± 0.05 | 0.233 ± 0.057735 |
| group | | | |
| 50 μg/mL BL21-MRP003 | 0.167 ± 0.175594 | 0.267 ± 0.152753 | 0.317 ± 0.152753 |
| group | | | |
| 100 μg/mL BL21-MRP003 | 0.217 ± 0.152753 | 0.500 ± 0.100 | 0.517 ± 0.104083 |
| group | | | |
| 200 μg/mL BL21-MRP003 | 0.717 ± 0.028868 | 0.983 ± 0.028868 | 0.983 ± 0.028868 |
| group | | | |
| 12.5 μg/mL BL21-MRP004 | 0.133 ± 0.076376 | 0.250 ± 0.050 | 0.250 ± 0.050 |
| group | | | |
| 25 μg/mL BL21-MRP004 | 0.067 ± 0.028868 | 0.117 ± 0.028868 | 0.167 ± 0.076376 |
| group | | | |
| 50 μg/mL BL21-MRP004 | 0.083 ± 0.028868 | 0.200 ± 0.086603 | 0.217 ± 0.11547 |
| group | | | |
| 100 μg/mL BL21-MRP004 | 0.017 ± 0.028868 | 0.150 ± 0.050 | 0.150 ± 0.050 |
| group | | | |
| 200 μg/mL BL21-MRP004 | 0.100 ± 0.050 | 0.183 ± 0.104083 | 0.250 ± 0.100 |
| group | | | |
| 12.5 μg/mL BL21-MRP005 | 0.067 ± 0.057735 | 0.150 ± 0.050 | 0.167 ± 0.029 |
| group | | | |
| 25 μg/mL BL21-MRP005 | 0.050 ± 0.050 | 0.183 ± 0.161 | 0.233 ± 0.161 |
| group | | | |
| 50 μg/mL BL21-MRP005 | 0.100 ± 1.7E−17 | 0.283 ± 0.029 | 0.300 ± 0.000 |
| group | | | |
| 100 μg/mL BL21-MRP005 | 0.067 ± 0.076376 | 0.117 ± 0.076 | 0.117 ± 0.076 |
| group | | | |
| 200 μg/mL BL21-MRP005 | 0.033 ± 0.057735 | 0.150 ± 0.087 | 0.183 ± 0.058 |
| group | | | |

### Example 2. Cultivation of Plant Bug-Resistant Transgenic Cotton

I. Construction of Plant Expression Vector

The DNA sequence between the HindIII and EcoRI recognition sites (recognition sequences) of the expression vector pCambia2301 was replaced by the DNA sequence as shown in SEQ ID No.1, and the remaining DNA sequences were kept unchanged, to obtain a recombinant vector pCambia2301-35S-MRP001-NOS (partial structure of which is schematically shown in FIG. 2). SEQ ID No.1 is composed of 2093 nucleotides, in which the positions 1 to 860 are the DNA sequence of the CaMV35S promoter, the positions 896 to 1816 are a DNA coding sequence of the MRP001 protein, and the positions 1830 to 2093 are the DNA sequence of the NOS terminator.

It was confirmed by identification through cleavage (FIG. 3) and DNA sequencing that the nucleotide sequence of the MRP001 gene in pCambia2301-35S-MRP001-NOS was the nucleotide sequence as shown in positions 896 to 1816 of SEQ ID No.1. The pCambia2301-35S-MRP001-NOS could express the MRP001 protein of SEQ ID No.6, and the sequence of positions 896 to 1816 of SEQ ID No.1 was the MRP001 protein encoding sequence.

II. Cultivation of Plant Bug-Resistant Transgenic Cotton

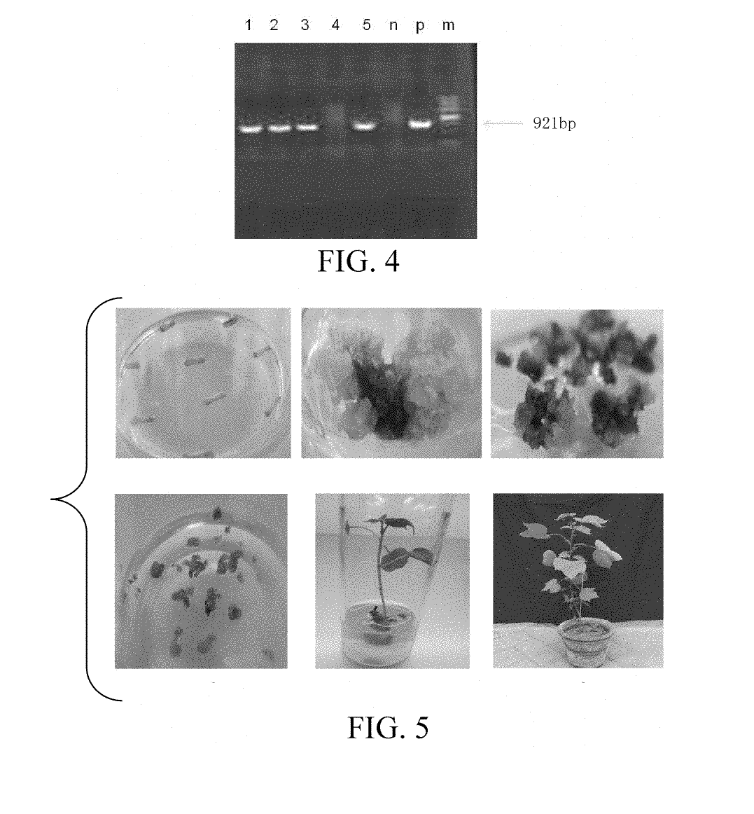

The Agrobacterium LBA4404 was transformed with the pCambia2301-35S-MRP001-NOS, to obtain a recombinant Agrobacterium containing pCambia2301-35S-MRP001-NOS, which was designated as LBA4404/MRP001. Following the method described in a literature (Firoozabady E, DeBoer D L, Merlo D J, et al. Transformation of cotton (Gossypium hirsutum L.) by Agrobacterium tumefaciens and regeneration of transgenic plants[J]. Plant Molecular Biology, 1987, 10(2): 105-116.), the hypocotyl of the CCRI 24 cotton was transformed with LBA4404/MRP001 by Agrobacterium-mediated transformation, to transform the MRP001 gene into the genome of cotton, followed by kanamycin screening, regeneration, and then grafting of transgenic cotton. When the transformed plant was grown to have 7-8 leaves, DNA was extracted from the leaves. PCR amplification was performed with the DNA as a template and C004-11-f (5′-CAGGGTGGTGATTTTGGTTA-3′) and C004-11-r (5′-CGGAGCCATTTCAGTGACATT-3′) as primers, to preliminarily detect the insertion of a foreign gene. A PCR product of about 920 bp was obtained (FIG. 4). After sequencing, the PCR product is shown to be 921 bp, and has a nucleotide sequence that is the nucleotide sequence as shown in positions 896 to 1816 of SEQ ID No.1. The DNA molecule of the nucleotide sequence is a MRP001 protein coding sequence, and the protein encoded by which is designated as MRP001 protein, which has an amino acid sequence of SEQ ID No.6. The transformed plant with the resultant 921 bp PCR product is designated as MRP001 transgenic cotton.

An empty vector pCambia2301 was transformed into the genome of CCRI 24 cotton following the same method, to obtain a transgenic cotton containing the empty vector, which is designated as empty vector transformed cotton.

III. Anti-Plant Bug Effect of Transgenic Cotton

Test material: The non-transgenic CCRI 24 was used as control 1, the empty vector transformed cotton of generation T₀ obtained in Step II was used as control 2, and the MRP001 transgenic cotton of generation T₀ obtained in Step II was used as test material.

The experiment was repeated three times. The experimental method was as follows. The test was carried out in a screen cage that was 20 meters long, 2.5 meters wide, and 1.8 meters high, and a nylon mesh of 80 meshes was covered outside of the screen cage, to prevent the assess of pests and natural enemies. 10 plants of the non-transgenic CCRI 24 (control group 1), the MRP001 transgenic cotton of generation T₀ obtained in Step II (control group 2), and the empty vector transformed cotton of generation T₀ obtained in Step II were respectively grown in the screen cage at a row spacing of 0.80 m and a plant spacing of 0.25 m, and the aphids was controlled 1-2 times during the seedling stage.

The adult Apolygus lucorum was released at a density of 2 bugs/square meter in the blossom period of cotton, and the released Apolygus lucorum was natural populations collected from the field, which were adults with high activity and manually fed with garden beans in door.

At day 6 after insect inoculation, the damage to leaves and squares by Apolygus lucorum in the control groups and test groups was investigated following the method described in Chinese Patent Application No. 201010284395.4 entitled Method for identifying plant bug-resistant cotton. The injury level of the leaves and squares were recorded. The symptoms of leaf injury include uneven distribution of holes in the leaf, and occurrence of brown or black spots. The symptoms of square injury include the occurrence of black pricks on the bracteal leaf of square, and even black square in a serious case. The determination criteria of leaf injury level include: level 0, no symptoms of leaf injury; level 1, slight leaf injury, where the injured area <5%; level 2, moderate leaf injury, where 5%<the injured area ≤20%; level 3, serious leaf injury, where 20%<the injured area ≤50%; and level 4, the same leaf injury as that in the control group, where the injured area >50%.

The leaf injury index, the decline in leaf injury index, the square injured rate, and the decline in square injured rate are calculated. The leaf injury index is an average of the injury levels of 5 leaves at the top of each cotton plant. The decline in leaf injury index is calculated according to Formula I:

$$Decline inleaf injury index \% = \frac{ \begin{matrix}Leaf injury index in control group - \\ Leaf injury index in test group\end{matrix}}{Leaf injury index in control group} \times 100 \% \qquad Formula I$$

The square injured rate is calculated according to Formula

$$Square injured rate \% = \frac{Number of injured squares}{\begin{matrix}Number of injured squares + \\ Number of healthy squares\end{matrix}} \times 100 \% \qquad Formula II$$

The decline in square injured rate is calculated according to Formula III:

$$Decline in square injured rate \% = \frac{\begin{matrix}Square injured rate in control group - \\ Square injured rate in test group\end{matrix}}{Square injured rate in control group} \times 100 \% \qquad Formula III$$

The test results are shown in Table 2. The leaf injury index of the MRP001 transgenic cotton is 0.35, and the decline in leaf injury index is 84.75%. The square injured rate is 2.89%, and the decline square injured rate is 88.89%. Compared with the cotton in control groups 1 and 2, the MRP001 transgenic cotton is highly resistant to Apolygus lucorum.

TABLE 2
Test results of plant bug-resistant cotton

| | | | | Decline in | Identification |
|---|---|---|---|---|---|
| Material | Leaf injury | Decline in leaf | Square injured | square injured | of resistance |
| Name | index | injury index (%) | rate (%) | rate (%) | to plant bug |
| Control | 2.20 ± 0.021 | 0.00 ± 0.000 | 24.65 ± 0.014 | 0.00 ± 0.000 | Not |
| group 1 | | | | | resistant |
| Control | 2.40 ± 0.005 | 0.00 ± 0.000 | 27.55 ± 0.102 | 0.00 ± 0.000 | Not |
| group 2 | | | | | resistant |
| MRP001 | 0.35 ± 0.012 | 84.75 ± 0.001 | 2.89 ± 0.002 | 88.89 ± 0.877 | Highly |
| transgenic | | | | | resistant |
| cotton | | | | | |

The experiment where the cotton bollworm is fed with a liquid feed containing the same concentration of MRP001 protein as above under the same experimental conditions shows that no death occurs to the cotton bollworm fed with the 200 μg/mL MRP001 protein liquid feed, so that the MRP001 protein is not resistant to cotton bollworm, and the MRP001 transgenic cotton is not resistant to cotton bollworm either, indicating that the MRP001 protein has only anti-plant bug activity, and no anti-cotton bollworm activity.

For the purpose of developing a new variety of cotton bollworm, the Cry51Aa1 gene is artificially mutated by the present inventors, to enhance the insect-resistance, and provide a reserve of insect-resistant gene of cotton bollworm. In this regard, a gene encoding the protein is artificially synthesized, and genes having differently mutated sites are designed according to the gene sequence. The protein is expressed in a prokaryotic expression system such as E. Coli. Through foliar spray of the protein, a mutated gene for enhancing the resistance to cotton bollworm is sought in a field environment. However, the result is poor, and no proteins having increased killing effect on cotton bollworm are found. The plant bug is fed with the same proteins, and it is found that the protein expressed by one of the mutated gene has a good plant bug killing effect.

### INDUSTRIAL APPLICATION

Considering that the MRP001 protein and biological materials associated therewith have resistance to plant bug, they are useful in the preparation of biological anti-insect agent containing the protein, and in the cultivation of insect-resistant transgenic cotton, fruits, tea, rice, vegetables and other crops, thereby reducing the application of pesticides and alleviating the environmental pollution, and thus being of great economic value and broad application prospect.

1. A method, comprising:
regulating plant resistance to insects with a protein;
preparing insect-resistant plant products with the protein;
regulating plant resistance to insects with biological materials associated with the protein;
preparing insect-resistant plant products with the biological materials associated with the protein; and/or
cultivating insect-resistant plants with the biological materials associated with the protein,
the protein is a protein of A1) or A2):
A1) a protein having an amino acid sequence as shown in SEQ ID No.6; and
A2) a protein having insect resistance derived from A1) by substituting, and/or deleting, and/or adding one or more amino acid residues in the amino acid sequence of the protein of A1); the biological materials are at least one of B1) to B10):
B1) a nucleic acid molecule encoding the protein;
B2) an expression cassette comprising the nucleic acid molecule of B1);
B3) a recombinant vector comprising the nucleic acid molecule of B1), or a recombinant vector comprising the expression cassette of B2);
B4) a recombinant microorganism comprising the nucleic acid molecule of B1), or a recombinant microorganism comprising the expression cassette of B2), or a recombinant microorganism comprising the recombinant vector of B3);
B5) a transgenic plant cell line comprising the nucleic acid molecule of B1), or a transgenic plant cell line comprising the expression cassette of B2), or a transgenic plant cell line comprising the recombinant vector of B3);
B6) a transgenic plant tissue comprising the nucleic acid molecule of B1), or a transgenic plant tissue comprising the expression cassette of B2), or a transgenic plant tissue comprising the recombinant vector of B3);
B7) a transgenic plant organ comprising the nucleic acid molecule of B1), or a transgenic plant organ comprising the expression cassette of B2), or a transgenic plant organ comprising the recombinant vector of B3);
B8) a transgenic plant comprising the nucleic acid molecule of B1), or a transgenic plant comprising the expression cassette of B2), or a transgenic plant comprising the recombinant vector of B3);
B9) a tissue culture produced from regenerable cells of the transgenic plant of B8); and
B10) a protoplast produced from the tissue culture of B9).

2. The method according to claim 1, wherein the plant is a host of plant bug.

3. The method according to claim 2, wherein the host of plant bug is cotton.

4. The method according to claim 1, wherein the insect is plant bug.

5. A botanical insecticide, wherein the botanical insecticide comprises the protein according to claim 1.

6. The botanical insecticide according to claim 5, wherein the plant is a host of plant bug.

7. The botanical insecticide according to claim 6, wherein the host of plant bug is cotton.

8. The botanical insecticide according to claim 5, wherein the insect is plant bug.

9. A method for preparing the protein according to claim 1, comprising introducing a gene encoding the protein according to claim 1, to a recipient cell to obtain a recombinant cell, and culturing the recombinant cell, to obtain the protein according to claim 1; wherein the recipient cell is a microorganism cell, a non-human animal cell, or a plant cell.

10. The method according to claim 9, wherein the gene encoding the protein according to claim 1 is a nucleic acid molecule of B1a), B1b), B1c) or B1d):
B1a) a DNA or cDNA molecule having a coding sequence as shown in positions 896 to 1816 of SEQ ID No. 1;
B1b) a cDNA or genomic DNA molecule that is 75% or more identical to the nucleotide sequence defined in B1a) and encodes the protein according to claim 1;
B1c) a cDNA or genomic DNA molecule that hybridizes to the nucleotide sequence defined in B1a) under stringent conditions and encodes the protein according to claim 1; or
B1d) a DNA molecule that is reversely complementary to the DNA molecule of B1a), B1b), or B1c).

11. A method for cultivating an insect-resistant transgenic plant, comprising introducing a gene encoding the protein according to claim 1 to a recipient plant, to obtain an insect-resistant transgenic plant.

12. The method according to claim 11, wherein the gene encoding the protein according to claim 1 is a nucleic acid molecule of B1a), B1b), B1c) or B1d):
B1a) a DNA or cDNA molecule having a coding sequence as shown in positions 896 to 1816 of SEQ ID No. 1;
B1b) a cDNA or genomic DNA molecule that is 75% or more identical to the nucleotide sequence defined in B1a) and encodes the protein according to claim 1;
B1c) a cDNA or genomic DNA molecule that hybridizes to the nucleotide sequence defined in B1a) under stringent conditions and encodes the protein according to claim 1; or
B1d) a DNA molecule that is reversely complementary to the DNA molecule of B1a), B1b), or B1c).

13. The method according to claim 11, wherein the plant is a host of plant bug.

14. The method according to claim 13, wherein the host of plant bug is cotton.

15. The method according to claim 11, wherein the insect is plant bug.

16. A protein, comprising: a protein of A1) or A2):
A1) a protein having an amino acid sequence as shown in SEQ ID No.6; and
A2) a protein having insect resistance derived from A1) by substituting, and/or deleting, and/or adding one or more amino acid residues in the amino acid sequence of the protein of A1).

17. A protein associated biological material, comprising: at least one of B1) to B10):
B1) a nucleic acid molecule encoding the protein according to claim 16;
B2) an expression cassette comprising the nucleic acid molecule of B1);
B3) a recombinant vector comprising the nucleic acid molecule of B1), or a recombinant vector comprising the expression cassette of B2);
B4) a recombinant microorganism comprising the nucleic acid molecule of B1), or a recombinant microorganism comprising the expression cassette of B2), or a recombinant microorganism comprising the recombinant vector of B3);
B5) a transgenic plant cell line comprising the nucleic acid molecule of B1), or a transgenic plant cell line comprising the expression cassette of B2), or a transgenic plant cell line comprising the recombinant vector of B3);
B6) a transgenic plant tissue comprising the nucleic acid molecule of B1), or a transgenic plant tissue comprising the expression cassette of B2), or a transgenic plant tissue comprising the recombinant vector of B3);
B7) a transgenic plant organ comprising the nucleic acid molecule of B1), or a transgenic plant organ comprising the expression cassette of B2), or a transgenic plant organ comprising the recombinant vector of B3);
B8) a transgenic plant comprising the nucleic acid molecule of B1), or a transgenic plant comprising the expression cassette of B2), or a transgenic plant comprising the recombinant vector of B3);
B9) a tissue culture produced from regenerable cells of the transgenic plant of B8); and
B10) a protoplast produced from the tissue culture of B9).

18. The protein associated biological material according to claim 17, wherein the nucleic acid molecule of B1) is a nucleic acid molecule of B1a), B1b), B1c) or B1d):
B1a) a DNA or cDNA molecule having a coding sequence as shown in positions 896 to 1816 of SEQ ID No. 1;
B1b) a cDNA or genomic DNA molecule that is 75% or more identical to the nucleotide sequence defined in B1a) and encodes the protein according to claim 1;
B1c) a cDNA or genomic DNA molecule that hybridizes to the nucleotide sequence defined in B1a) under stringent conditions and encodes the protein according to claim 1; or
B1d) a DNA molecule that is reversely complementary to the DNA molecule of B1a), B1b), or B1c).


SEQUENCE LISTING (8 sequences)

SEQ ID NO: 1 - DNA, 2093 bp, artificial sequence
  pET28a-MRP001 recombinant vector
gcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagaggatctactagtcatatggattggcgcgccatggccattcttgatttaaaatctcttgtgctcaatgcaattaattactggggtccaaagaacaataatggtatccagggtggtgattttggttatcctattagcgaaaaacaaattgatacaagtattattacgtcaacccatccaaggttgataccccatgatctgaccattccacaaaacttggagacaattttcacaaccactcaggtattaactaataacactgatttgcagcaatcccaaacagtgtccttcgctaagaagactactaccaccactgctacctccacaacgaacggctggacagaaggcggaaaaatttcagatacgttggaggaaaaggtttcagtttcaattccctttatcggggaaggtggagggaagaatagcaccaccattgaggctaacttcgctcataatagttccaccaccacagcccaacaggccagcactgacatcgaatggaacattagccaaccagtgctcgtgcctcccagaaagcaggtagttgcaacactcgttataatgggcggtaatttcactatccctatggacctgatgacaactatagattcaacagagcactattctggctatcctatacttacttggataagtagcccagataactcttataacgggccgtttatgtcctggtacttcgctaattggcctaacttaccaagtggatttgggcctttgaactccgacaatacggttacttatactggatctgtggtttctcaagtctctgctggagtttacgcaaccgtcagatttgaccaatacgatatccacaatcttcgaactatcgagaaaacatggtacgcacgtcacgccacactccataacggaaagaaaatctccattaacaatgtcactgaaatggctccgacttcacctatcaaaaccaattaaggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgg

SEQ ID NO: 2 - DNA, 930 bp, artificial sequence
  MRP002 protein coding sequence
atgatcttccttgccattcttgatttaaaatctcttgtgctcaatgcaattaattactggggtccaaagaacaataatggtatccagggtggtgattttggttatcctattagcgaaaaacaaattgatacaagtattattacgtcaacccatccaaggttgataccccatgatctgaccattccacaaaacttggagacaattttcacaaccactcaggtattaactaataacactgatttgcagcaatcccaaacagtgtccttcgctaagaagactactaccaccacttctacctccacaacgaacggctggacagaaggcggaaaaatttcagatacgttggaggaaaaggtttcagtttcaattccctttatcggggaaggtggagggaagaatagcaccaccattgaggctaacttcgctcataatagttccaccaccacatttcaacaggccagcactgacatcgaatggaacattagccaaccagtgctcgtgcctcccagaaagcaggtagttgcaacactcgttataatgggcggtaatttcactatccctatggacctgatgacaactatagattcaacagagcactattctggctatcctatacttacttggataagtagcccagataactcttataacgggccgtttatgtcctggtacttcgctaattggcctaacttaccaagtggatttgggcctttgaactccgacaatacggttacttatactggatctgtggtttctcaagtctctgctggagtttacgcaaccgtcagatttgaccaatacgatatccacaatcttcgaactatcgagaaaacatggtacgcacgtcacgccacactccataacggaaagaaaatctccattaacaatgtcactgaaatggctccgacttcacctatcaaaaccaattaa

SEQ ID NO: 3 - DNA, 930 bp, artificial sequence
  MRP003 protein coding sequence
atgatcttccttgccattcttgatttaaaatctcttgtgctcaatgcaattaattactggggtccaaagtacaataatggtatccagggtggtgattttggttatcctattagcgaaaaacaaattgatacaagtattattacgtcaacccatccaaggttgataccccatgatctgaccattccacaaaacttggagacaattttcacaaccactcaggtattaactaataacactgatttgcagcaatcccaaacagtgtccttcgctaagaagactactaccaccacttctacctccacaacgaacggctggacagaaggcggaaaaatttcagatacgttggaggaaaaggtttcagtttcaattccctttatcggggaaggtggagggaagaatagcaccaccattgaggctaacttcgctcataatagttccaccaccacatttcaacaggccagcactgacatcgaatggaacattagccaaccagtgctcgtgcctcccagaaagcaggtagttgcaacactcgttataatgggcggtaatttcactatccctatggacctgatgacaactatagattcaacagagcactattctggctatcctatacttacttggataagtagcccagataactcttataacgggccgtttatgtcctggtacttcgctaattggcctaacttaccaagtggatttgggcctttgaactccgacaatacggttacttatactggatctgtggtttctcaagtctctgctggagtttacgcaaccgtcagatttgaccaatacgatatccacaatcttcgaactatcgagaaaacatggtacgcacgtcacgccacactccataacggaaagaaaatctccattaacaatgtcactgaaatggctccgacttcacctatcaaaaccaattaa

SEQ ID NO: 4 - DNA, 930 bp, artificial sequence
  MRP004 protein coding sequence
atgatcttccttgccattcttgatttaaaatctcttgtgctcaatgcaattaattactggggtccaaagaacaataatggtatccagggtggtgattttggttatcctattagcgaaaaacaaattgatacaagtattattacgtcaacccatccaaggttgataccccatgatctgaccattccacaaaacttggagacaattttcacaaccactcaggtattaactaataacactgatttgcagcaatcccaaacagtgtccttcgctaagaagactactaccaccacttctacctccacaacgaacggctggacagaacgcggaaaaatttcagatacgttggaggaaaaggtttcagtttcaattccctttatcggggaaggtggagggaagaatagcaccaccattgaggctaacttcgctcataatagttccaccaccacatttcaacaggccagcactgacatcgaatggaacattagccaaccagtgctcgtgcctcccagaaagcaggtagttgcaacactcgttataatgggcggtaatttcactatccctatggacctgatgacaactatagattcaacagagcactattctggctatcctatacttacttggataagtagcccagataactcttataacgggccgtttatgtcctggtacttcgctaattggcctaacttaccaagtggatttgggcctttgaactccgacaatacggttacttatactggatctgtggtttctcaagtctctgctggagtttacgcaaccgtcagatttgaccaatacgatatccacaatcttcgaactatcgagaaaacatggtacgcacgtcacgccacactccataacggaaagaaaatctccattaacaatgtcactgaaatggctccgacttcacctatcaaaaccaattaa

SEQ ID NO: 5 - DNA, 930 bp, artificial sequence
  MRP005 protein coding sequence
atgatcttccttgccattcttgatttaaaatctcttgtgctcaatgcaattaattactggggtccaaagaacaataatggtatccagggtggtgattttggttatcctattagcgaaaaacaaattgatacaagtattattacgtcaacccatccaaggttgataccccatgatctgaccattccacaaaacttggagacaattttcacaaccactcaggtattaactaataacactgatttgcagcaatcccaaacagtgtccttcgctaagaagactactaccaccacttctacctccacaacgaacggctggacagaaggcggaaaaatttcagatacgttggaggaaaaggtttcagtttcaattccctttatcggggaaggtggagggaagaatagcaccaccattgaggctaacttcgctcataatagttccaccaccacatttcaacaggccagcactgacatcgaatggaacattagccaatcagtgctcgtgcctcccagaaagcaggtagttgcaacactcgttataatgggcggtaatttcactatccctatggacctgatgacaactatagattcaacagagcactattctggctatcctatacttacttggataagtagcccagataactcttataacgggccgtttatgtcctggtacttcgctaattggcctaacttaccaagtggatttgggcctttgaactccgacaatacggttacttatactggatctgtggtttctcaagtctctgctggagtttacgcaaccgtcagatttgaccaatacgatatccacaatcttcgaactatcgagaaaacatggtacgcacgtcacgccacactccataacggaaagaaaatctccattaacaatgtcactgaaatggctccgacttcacctatcaaaaccaattaa

SEQ ID NO: 6 - protein, 306 aa, artificial sequence
  MRP001 protein
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTAQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 7 - DNA, 20 bp, artificial sequence
  C004-11-f primer
cagggtggtgattttggtta

SEQ ID NO: 8 - DNA, 21 bp, artificial sequence
  C004-11-r primer
cggagccatttcagtgacatt